(19) United States
(12) Patent Application Publication
(10) Pub. No.: US 2017/0074847 A1
(43) Pub. Date: Mar. 16, 2017

(54) Fresh Check Monitoring System

(71) Applicants: Krysten Nyckole Smith, Anadarko, OK (US); Clarence Neal Stidham, Anadarko, OK (US)

(72) Inventors: Krysten Nyckole Smith, Anadarko, OK (US); Clarence Neal Stidham, Anadarko, OK (US)

(21) Appl. No.: 14/854,337

(22) Filed: Sep. 15, 2015

(51) Int. Cl. G01N 33/02; C12Q 1/04

(52) U.S. Cl. CPC G01N 33/02; C12Q 1/04

(57) ABSTRACT

A device for monitoring the freshness of food items is provided; made up of plastic and thermos-measuring product. It detects mold or bad gases from the stored food when it reacts with the gasses released by microorganisms during food spoilage or mold. It provides visual indication to the user about the freshness of food items by changing colors. When it stays green the food is safe to eat and when food becomes unsafe it shows red indication to the user. It can be placed in a redesigned Tupperware lid, cup, plates, zip lock bag, or directly built into all prepackaged organic food products; such as prepackaged meat, milk jugs, juices, animal feed, plant seed or anything with an expiration date. It is designed for one time use only.

### CROSS REFERENCE TO RELATED APPLICATIONS

Not applicable

### FEDERALLY SPONSORED RESEARCH AND DEVELOPMENT

Not applicable.

### MICROFICHE

Not applicable

### BACKGROUND OF THE INVENTION

(1) Field of the Invention

The disclosures made herein relate generally to device for detecting mold or bad gases and, more particularly, to methods and devices for detecting and/or indicating spoilage of food items.

(2) Background of the Invention

It is well known that many food items, if not all food items, are subject to spoilage. Consumption of such spoiled food items can cause serious illness. In some instances (e.g., weak immune system, excessive consumption, failure to seek medical assistance, etc.), consumption of such spoiled food items can be fatal or cause permanent disability.

Modern food products come in a variety of forms including fresh, frozen, and freeze-dried. A major factor in the tastiness of prepared foods is the freshness of the food products or ingredients. Many such food products require refrigeration or must be maintained in a frozen condition to best preserve freshness. However, refrigerated food products gradually lose freshness over time and are no longer usable after periods of time ranging from a few days to several weeks. Likewise, even frozen food products become un-useable, though typically over longer periods of time ranging from weeks to months. For example, ice cream and sherbet gradually separate into their component parts and are un-usable after a period of time from about two to three months. Meats packaged in shrink wrap plastic film lose moisture and become freezer burned after a period of time from about one to two months. While some food products have expiration dates printed on their packaging, many do not, or the expiration date is not readily visible.

Food that is spoiled to an excessive degree is typically fairly easy to identify either by sight, smell and/or taste. The ability to see, smell and/or taste indication(s) that a food item is excessively spoiled actually makes such excessively spoiled food item less susceptible to being unintentionally consumed. To the contrary, a food item that is at the onset of spoilage or early stages of being spoiled is very susceptible to unintentional consumption because it may not be immediately apparent though sight, smell or taste that the food item is at the onset of spoiling or is spoiled. Because of this, a person may actually consume large quantities of such food items, which increases the potential for serious illness, fatality and/or disability.

Contributing to the unintentional consumption of spoiled food items is the wide variety of food storage systems that are commercially-available to consumers. Examples of such commercially-available food storage systems include, but are not limited to, Zip-Loc™ brand plastic bags, Rubbermaid™ brand containers and Tupperware™ brand containers. In most countries, it is common practice for consumers to store remaining portions of food items (e.g., consumer-prepared left-overs, bulk packaged left-overs, etc.) in commercially-available food storage systems. The lack of preservatives in certain food items, inadequate refrigeration temperature, exposure to air, storage duration and other such variables make food items stored in such commercially-available food storage systems particularly susceptible to spoilage.

So the current invention proposes a device which can detect mold and bad gases from the stored food. Now user can know about the freshness of food without smelling and tasting the food. It provides visual indication to the user about the freshness of food. If it detects any kind of mold and bad gas inside the Tupperware then it will show red indication to the user.

### SUMMARY OF THE INVENTION

In the view of the foregoing disadvantages inherent in the known types of fresh food monitoring devices now present in the prior art, the present invention provides an improved device for monitoring the freshness of food. As such, the general purpose of the present invention, which will be described subsequently in greater detail, is to provide a new and improved device for monitoring the freshness of food, which has all the advantages of the prior art and none of the disadvantages.

A primary object of the present invention is to provide a device which can detect mold and bad gases which comes from the food after spoilage as per the preferred embodiment of the present invention.

It is therefore an object of the present invention to provide a fresh food monitoring device which can fit to any product which has food item as per the preferred embodiment of the present invention.

It is further object of the present invention to provide a fresh food monitoring device which prevents user from illness which happens due to eating of spoiled food as per the preferred embodiment of the present invention.

It is therefore an object of the present invention to provide a fresh food monitoring device which provides visual indication to the user about the food condition as per the preferred embodiment of the present invention.

It is further object of the present invention to provide a fresh food monitoring device which provides red indication after expiry of food item as per the preferred embodiment of the present invention.

It is therefore an object of the present invention to provide a fresh food monitoring device which can replace the need of expiration date stamps as per the preferred embodiment of the present invention.

It is further object of the present invention to provide a fresh food monitoring device which can work in any environment as per the preferred embodiment of the present invention.

It is still further object of the present invention to provide a fresh food monitoring device which is of plastic and smaller in size as per the preferred embodiment of the present invention.

Another object of the present invention is to provide a fresh food monitoring device that will be economical to manufacture and easy to implement as per the preferred embodiment of the present invention.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty which characterize the invention, are pointed out with particularity in the disclosure. For a better understanding of the invention, its operating advantages and the specific objects attained by its uses, reference should be had to the accompanying descriptive matter in which there are illustrated preferred embodiments of the invention.

Accordingly, the present disclosure includes features and advantages which are believed to enable it to advance the art of placing items on carriers. Characteristics and advantages of the present disclosure described above and additional features and benefits will be readily apparent to those skilled in the art upon consideration of the following detailed description and referring to the accompanying drawings.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 shows the perspective view of preferred embodiment.

### DETAILED DESCRIPTION OF THE INVENTION

In the following detailed description many embodiments are described as per the invention. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description is, therefore, not to be taken in a limiting sense, and the scope of the present invention is defined by the appended description and their equivalents.

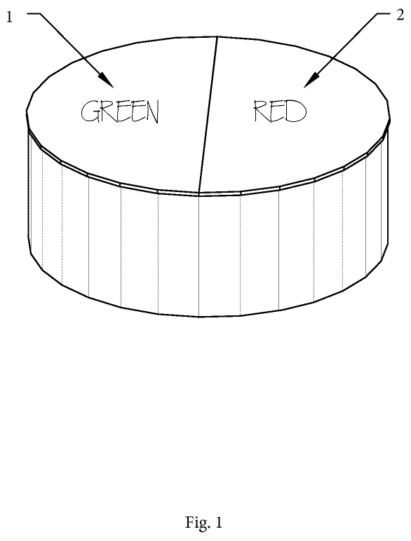

The present invention is a device for monitoring the freshness of food items FIG. 1. It detects mold or bad gases from the stored food. It reacts to the presence of gases released by microorganisms during food spoilage or mold. It provides visual indication to the user about the freshness of food items. It stays green 1 when the food is safe to eat and when food becomes unsafe it shows red 2 indication to the user. It is made up of plastic and thermo measuring product. It can be placed in a redesigned Tupperware lid, cup, plates, zip lock bag, or directly built into all prepackaged organic food products; such as prepackaged meat, milk jugs, juices, animal feed, plant seed or anything with an expiration date. Users cannot use it again and again. It is only for one time use.

It is to be understood that the above description is intended to be illustrative, and not restrictive. For example, the above-discussed embodiments may be used in combination with each other. Many other embodiments will be apparent to those of skill in the art upon reviewing the above description.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention.

We claim:

1. A device for indicating the condition of stored food comprising;
means for storage of food material;
means for detecting mold gases; and
means for visual indication.

2. The device for indicating the condition of stored food of claim 1, wherein said means for storage of food material is a zip lock bag or like.

3. The device for indicating the condition of stored food of claim 1, wherein said means for storage is directly built prepackaged organic food product.

4. The device for indicating the condition of stored food of claim 1, wherein said means for detecting mold gases is placed inside said means for storage of food material.

5. The device for indicating the condition of stored food of claim 1, wherein said means for visual indication indicates the freshness of stored food.

6. The device for indicating the condition of stored food of claim 1, wherein said means for visual indication stays green when stored food is safe to eat.

7. The device for indicating the condition of stored food of claim 5, wherein means for visual indication slowly turns red as stored food becomes unsafe for consumption.

8. The device for indicating the condition of stored food of claim 1, wherein said device keeps track of expiration of food items.

9. A method for indicating the condition of stored food comprising steps of
measuring rise in temperature inside means for storage of food material;
attaching a piece of plastic on inside of means for storage of food material;
detecting presence of foul gases released by microorganisms during food spoilage or mold; and
indicating freshness of food material by, placing a piece of plastic inside means for storage of food material.

10. The method for indicating the condition of stored food of claim 9, wherein said means for storage of food material is a zip lock bag or like.

11. The method for indicating the condition of stored food of claim 9, wherein temperature inside said means for storage of food material is measured using a thermo-measuring material.

12. The method for indicating the condition of stored food of claim 9, wherein the color of said piece of plastic changes color to visually indicate freshness of stored food material.

13. The method for indicating the condition of stored food of claim 12, wherein green color of said piece of plastic indicates stored food material is safe for consumption.

14. The method for indicating the condition of stored food of claim 12, wherein color of said piece of plastic turns to red upon reacting with the gases released by microorganisms during food spoilage or mold.
